(19) United States
(12) Patent Application Publication
(10) Pub. No.: US 2022/0151735 A1
(43) Pub. Date: May 19, 2022

(54) SPLIT WIRELESS DETECTION MASK AND DETECTION METHODS THEREOF

(71) Applicant: CHENGHENG (SHANGHAI) HEALTH & MEDICAL DEVICES CO., LTD., Shanghai (CN)

(72) Inventor: Jiang YUE, Beijing (CN)

(21) Appl. No.: 17/440,535

(22) PCT Filed: Aug. 30, 2019
(86) PCT No.: PCT/CN2019/103653

§ 371 Date: Sep. 17, 2021

(30) Foreign Application Priority Data
Jul. 16, 2019 (CN) 201910641393.7

(51) Int. Cl. A61C 7/06

(52) U.S. Cl. CPC A61C 7/06; G06F 3/044

(57) ABSTRACT

Disclosed is a split wireless detection mask, including a mask and a base which can be connected to each other. The mask is wearable on a human body and configured for acquiring information of the human body. When the mask is connected to the base, the base charges the mask, the mask transmits the acquired information to the base, and then the base transmits the information to a cloud server. The mask includes a control unit, a detection unit, and a detection information transmitting unit. The detection unit detects information of the mask, and if the human body wears the mask, the detection unit stores the information in the control unit. The base includes a wireless transmission unit, a detection information receiving unit, and a power source unit. The detection information receiving unit receives data sent by the detection information transmitting unit.

### TECHNICAL FIELD

The present invention relates to a wearable device technology, particularly to a split wireless detection mask and detection methods thereof.

### BACKGROUND

The existing orthodontic treatment method is to use an appliance to a patient, such as a removable appliance, a functional appliance, a fixed appliance, a retainer, etc., by applying an appropriate “biological force” to the teeth, alveolar bones and jaw bones, a physiological movement is generated. This method is slow in use process and long in effect taking time.

The patent with the application number 201811341718.1 proposed by the applicant proposes an accelerated orthodontic device and an accelerated orthodontic method, wherein a mask method is employed to correct the teeth of a patient. However, the wearing information of users cannot be acquired, such as wearing length of time and the like. Due to the fact that the information feedback is not enough in time and the expression of some patients is unclear, the treatment method implemented and treatment results are biased, the accurate acquisition of data of the whole treatment method cannot be realized, the treatment regimen cannot be accurately improved, and therefore accurate customized correction of the patient is not facilitated.

### SUMMARY

The problem to be solved by the present invention is to overcome the above-mentioned drawbacks in existing technologies, and to provide a split wireless detection mask and detection methods thereof that can detect wearing information of a user, and transmit to a cloud server at an appropriate time.

The technical problem of the invention can be solved by the following technical scheme:

A split wireless detection mask, including a mask and a base that are split and can be connected to each other, where the mask is wearable on a human body and configured for acquiring wearing information of the human body, and when the mask is connected to the base, the base can charge the mask, the mask transmits the acquired wearing information to the base, and the base transmits the information to a cloud server.

Further, the mask includes a control unit, a detection unit, a power supply unit and a detection information transmitting unit, where the detection unit, the power supply unit and the detection information transmitting unit are electrically connected with the control unit respectively, the power supply unit charges the mask, and the wearing information is acquired by the detection unit.

Further, the base includes a wireless transmission unit, a power source unit and a detection information receiving unit, where the power source unit is connected with an external power source, the wireless transmission unit, the power source unit and the detection information receiving unit are electrically connected, and the detection information receiving unit is configured for receiving data sent by the detection information transmitting unit.

Further, the wearing information includes information about whether a human body is wearing a mask, information about length of time of the mask being worn and other information about the mask being worn.

Further, if the detection unit detects that a human body is wearing, the control unit starts timing, and when the detection unit detects that the human body has removed the mask, the control unit stops timing so as to detect the information about the length of time of the mask being worn by the human body.

Further, other information about the mask being worn includes information of correction effect for characterizing the mask being used as an orthodontic apparatus, including a wearing posture, and a stress between the mask and teeth.

Further, connection of the mask to the base is an electrical connection, and information transmission between the mask and the base adopts wired or wireless transmission; and the base and the cloud server achieve data transmission through a wireless transmission unit.

Further, a plug connector or a magnetic connector is configured as a connecting type between the mask and the base.

Further, the power supply unit further includes a charging unit.

Further, the detection unit is a capacitive touch sensing circuit.

Further, the capacitive touch sensing circuit adopts an RC charging detection principle, and the capacitive touch sensing circuit includes a plurality of resistors, a plurality of capacitors and a plurality of reserved contacts, and the reserved contacts include conductive metal sheets for detecting whether a human body is connected to the detection unit.

Further, when a human body contacts the reserved contacts to cause the capacitive touch sensing circuit to conduct, it is equivalent to connecting in a capacitor for applying a periodic charging voltage to the capacitive touch sensing circuit, and detecting the voltage across a capacitor, and when the time required for the voltage across the capacitor to reach a threshold is extended, it is indicated that the human body has contacted the detection unit.

Further, the control unit includes a controller, and the model of the controller is one of STM32F103C8T6 or ATmega8.

Further, the control unit includes a timing unit, and the timing unit sets a fixed time, and continuously transmits a signal to the control unit periodically at the fixed time, where the control unit detects whether there is human contact in the detection unit, and if the human body is already in contact, the control unit starts the counting by taking a transmission signal of the timing unit at the moment when the human body is in contact, and when the control unit cannot receive the signal of the human contact in the detection unit, the transmission signal of the timing unit at this time is configured as counting end.

Further, the control unit includes a memory unit, the memory unit stores information detected by the detection unit, and the memory unit is a non-volatile memory unit.

Further, a transmission mode of the wireless transmission unit is one or more of GSM, WiFi or Bluetooth.

The present invention further provides a method for acquiring length of time being worn by a human body utilizing the split wireless detection masks as mentioned above, where a detection unit in the mask is utilized to detect whether a human body is wearing the mask, wearing length of time or other wearing information; if the human body is already wearing, the control unit starts counting and if the human body has removed, the control unit stops counting, and the detection unit transmits and stores the information about the wearing length of time and other wearing information to the control unit; when the mask is fixedly connected with the base, data in the control unit is transmitted to the base, then to a cloud server through a wireless transmission unit in the base, achieving real-time acquisition and transmission of wearing information of the human body.

Compared with existing technologies, the beneficial effects of the present invention are as follows.

According to the present invention, a structural design of separating the data acquisition part and the data transmission part is adopted, the data acquisition part is installed in the mask, the data acquisition part can be controlled to a small volume, the miniaturization of the acquisition circuit is realized, and therefore the lightweight installation of the data acquisition part on the mask is realized. Meanwhile, the use of the power supply unit in the mask can realize long-time data acquisition, and the acquired data is stored in the internal non-volatile memory unit, for example, EEPROM or Flash Memory, so that the stability of data storage is guaranteed.

In the present invention, the data acquisition part and the data transmission part can be configured for charging and data transmission through a socket or a magnetic connector and the like to facilitate connection; when the mask of the data acquisition part is inactive, the mask can be connected to the base for charging; the base is provided with a power source unit and a wireless transmission unit; data in the mask can be transmitted to a cloud server through the base while charging the mask, so that uploading of the data is realized, and the doctor can conveniently grasp the wearing information about patients in time.

By adopting the split wireless detection mask provided by the present invention to acquire wearing information of a user, such as wearing length of time and the like, deviations of data in correction options caused by the wearing problem of the user can be reduced, the correction efficiency is improved, the correction period is shortened, and the accuracy of data is improved. Meanwhile, in the present invention, by real-time acquisition of the information about the length time being worn by the user and sending back to the cloud server at the appropriate time, data acquisition is realized more timely when compared with a return visit of a patient, the accuracy of the data is increased, to provide accurate data for orthodontists during treatment, and this can improve the whole treatment process in time, as well as the trust between patients and orthodontists.

### BRIEF DESCRIPTION OF DRAWINGS

FIG. 1 is a schematic diagram of a detection unit according to the present invention;

FIG. 2 is a graph of charging and discharge time of the detection unit according to the present invention when not in contact with a human body;

FIG. 3 is a control diagram of the charging and discharge time the detection unit according to the present invention before and after in contact with the human body;

FIG. 4 is an actual circuit diagram of the detection unit according to the present invention;

FIG. 5 is a block diagram of a split wireless detection mask according to the present invention.

### DETAILED DESCRIPTION

Hereinafter, the present invention will be described further with reference to the preferred embodiments and with reference to the accompanying drawings.

In addition, the various components on the drawing are enlarged (thick) or reduced (thin) for the convenience of understanding, but this practice is not intended to limit the scope of protection of the present invention.

Words importing the singular number include the plural and vice versa.

In the description of the embodiments of the present invention, it should be noted that, the orientation or positional relationship indicated by the terms “upper,” “lower,” “inner,” “outer,” etc., is based on the orientation or positional relationship shown in the figures, or the orientation or positional relationship conventionally placed when the product is used, is merely for the convenience of describing the present invention and the simplified description, and is not intended to indicate or imply that the indicated device or element must have a particular orientation, constructed and operated in a particular orientation, and therefore not to be construed as a limitation of the present invention. In addition, in the description of the present invention, in order to distinguish different cells, the present description uses words of first, second, etc., but these are not limited by the order of manufacture, nor are it to be understood as indicating or implying relative importance, which may differ from the following detailed description of the invention and the claims.

The wording in the description is used for the purpose of illustrating the embodiments of the present invention and are not intended to be limiting the present invention. It should also be noted that, unless expressly specified and defined otherwise, the terms “set,” “attached,” and “connected,” are to be construed broadly, for example, may be fixedly connected, may be detachably connected, or integrally connected; may be mechanically connected, may be directly attached, may also be indirectly connected by an intermediate medium, may be internal communication of the two elements. It will be understood by those skilled in the art that the foregoing is a specific meaning in the present invention.

The present invention provides a split wireless detection mask, including a data acquisition part and a data transmission part which are split and can be connected with each other, and the data acquisition part is installed in the mask, and the data transmission part is installed in the base, a detection unit is arranged in the mask to acquire the wearing information of users, such as information about whether a human body is wearing a mask, information about length of time of the mask being worn and other information about the mask being worn, and store data in a control unit in the mask; when the mask is inactive, the mask can be connected with the base, the base is internally provided with a power source unit and a wireless transmission unit, the mask can be charged through the base, meanwhile, the mask can transmit the acquired wearing information data to the base, and then transmit to a cloud server through a wireless transmission unit in the base to complete data acquisition and data transmission of the mask.

Because the mask in the present invention is used in the field of human orthodontics, other information about the mask being worn includes information of correction effect for characterizing the mask being used as an orthodontic apparatus, including a wearing posture, a stress between the mask and teeth, and the like. In the embodiment, the mask is configured for acquiring information about length of time being worn by a human body.

The mask includes a control unit, a detection unit, a power supply unit and a detection information transmitting unit; the detection unit, the power supply unit and the detection information transmitting unit are electrically connected with the control unit respectively; the power supply unit is electrically connected with the detection information transmitting unit; the power supply unit supplies power to the whole mask; the control unit is configured for processing the information acquired by the detection unit; the detection unit is embedded in the mask and realizes the detection of whether a human body is wearing or not by contacting the skin of the human body, and when the mask is worn on an inanimate object, the detection unit can identify whether it is worn by the human body due to the fact that the skin of the human body is not in contact.

In the embodiment of the invention, the control unit includes a controller, and the model of the controller is one of STM32F103C8T6 or ATmega8.

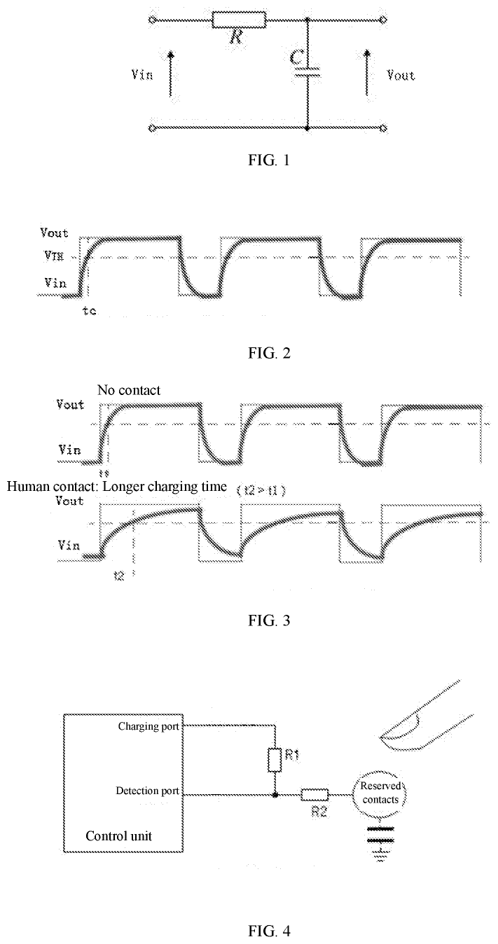

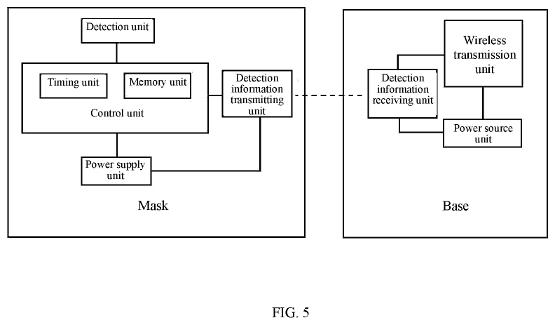

Specifically, the detection principle of the detection unit is a phase shift circuit, namely an RC charge detection circuit. As shown in FIG. 1, a capacitor and a resistor are arranged in the RC charging detection circuit. Meanwhile, reserved contacts are arranged in the circuit. When a human body contacts the reserved contacts, contact of the human body to the circuit reserved contacts is equivalent to connecting a capacitor, so that circuit parameters are changed. At this time, whether a human body is put in circuit or not can be detected by measuring the charging and discharge time of the capacitor in the RC circuit.

As shown in FIG. 1, under normal circumstances, a periodic charging voltage Vin is applied to the RC circuit to measure the Vout terminal to obtain a graph of the charging and discharge time of the capacitor as shown in FIG. 2, where the horizontal axis represents time, the longitudinal axis represents the voltage across the capacitor, and whether a human body is in contact or not can be judged by detecting the change in the time t_{c} of the capacitor from the charging start time to the Vout terminal to reach a certain threshold Vth. As shown in FIG. 3, when a human body contacts a circuit, the capacitor charging time becomes longer, that is, t2>t1.

The power supply unit is a rechargeable lithium battery, the voltage thereof is 3.7 V, the model number is 401235, and the threshold Vth measured by the Vout terminal is 2.5 V.

The circuit diagram of the detection unit in the mask is shown in FIG. 4, the control unit is connected with two ports, i.e. a charging port and a detection port, and the charging port and the detection port are respectively connected to the detection unit, the charging port charges the capacitor, and the detection port measures the voltage across the capacitor, where the resistance value of R1 is 1 megaohm, and is configured for controlling the charging time range, and the resistance value of R2 is 10 kilo ohms and is configured for reducing noise interference.

The detection unit is provided with reserved contacts for contact with a human body, where the reserved contacts are a conductive metal sheet, such as a stainless steel or copper sheet and the like, and the human body is in contact with the reserved contacts so as to be put in a circuit of the detection unit.

When the charging port is set, the detection port detects whether a threshold of a trigger voltage is reached, when the human body is in contact, the detection port detects the threshold of reaching the trigger voltage, and at this time, the control unit detects that the TC changes, indicating that the human body is in contact into the detection unit, thereby realizing the detection of human body contact.

The detection unit may also adopt a capacitive touch sensing circuit or a capacitive touch sensing chip to realize detection, the principle thereof is the same as the RC circuit, and transmit the detection result to the control unit.

The mask further includes a timing unit and a memory unit arranged on the control unit, where the timing unit is provided with a fixed time, so that the signal can be transmitted to the control unit in a timed manner, and the control unit re-checks whether voltage on the detection port in the detection unit reaches a threshold or not, if the threshold is reached, indicating that the human body is in contact, the control unit starts counting with the signal transmitted by the timing unit at this point, when the control unit checks that the voltage on the detection port in the detection unit does not reach the threshold, indicating that the human body is not in contact, the control unit stops counting with the signal transmitted by the timing unit at this point, to calculate the time from reaching the threshold to the time not reaching the threshold, that is, the length of time of the human body in contact into the detection unit, so that the length of time being worn by a user is detected, and the wearing length of time data is transmitted to the memory unit in the control unit, and the data is saved.

The memory unit is a non-volatile memory unit, such as an Electrically Erasable Programmable Read-Only Memory (EEPROM) or a Flash Memory.

Since the time interval set by the timing unit is the same, the accuracy of the wearing time measurement is the time set by the timing unit, the timing unit transmits a signal to the control unit every set time, the detection unit detects whether the human body is wearing, and transmits the signal to the control unit, so that the actual length of time being worn by users can be accurately detected, and the detection data can be stored in the memory unit.

The mask includes a power supply unit electrically connected with the control unit, and a detection information transmitting unit, where the power supply unit supplies power to the whole mask, the detection information transmitting unit is electrically connected with the power supply unit and the control unit respectively, and data in the memory unit is transmitted out of the mask through the detection information transmitting unit.

The base includes a power supply unit, a wireless transmission unit and a detection information receiving unit, where the wireless transmission unit and the detection information receiving unit are electrically connected with the power supply unit respectively, the power supply unit is connected with an external power socket to supply power to the base, and the wireless transmission unit and the detection information receiving unit are electrically connected.

The transmission mode of the wireless transmission unit can be one or more of GSM, WiFi or Bluetooth.

The detection information receiving unit and the detection information transmitting unit can be flexibly connected using such as a socket or a magnetic connector.

When the mask is inactive, the connection between the mask and the base can be realized through the connection between the detection information transmitting unit and the detection information receiving unit, the power supply unit can charge the power supply unit in the mask through two connectors, the storage unit in the mask can transmit the data to the wireless transmission unit through the two connectors, the wireless transmission unit transmits data to a cloud server, the data acquisition and transmission are completed, the data can facilitate an orthodontist to timely master the wearing information of a patient, and avoid affect the treatment of the orthodontist due to unclear expression of the patient or too long return visit time.

The present invention further provides a method for detecting the above-mentioned length of time of the mask being worn, where a detection unit in the mask is utilized to detect whether a human body is wearing the mask and if the human body is already wearing, the detection unit transmits a signal to a control unit in the mask, the control unit starts counting and if the human body has removed, the control unit stops counting, and the control unit detects the information about the length of time being worn by the human body, and saves the information in the control unit; when the detection information transmitting unit and the detection information receiving unit are fixedly connected, data in the control unit is transmitted to the base, then to a cloud server through a wireless transmission unit, achieving real-time acquisition and transmission of the information about the length of time being worn by the human body. Detection of other wearing information by the mask can obtain related information of orthodontic effects by arranging a pressure sensor, an ultrasonic sensor or an image sensor according to needs in a detection unit of the mask.

The above has been described in detail with respect to the specific embodiments of the present invention. It will be apparent to those skilled in the art that various modifications and adaptations may be made to the present invention without departing from the principles of the invention, which are also intended to be within the scope of the appended claims.

1. A split wireless detection mask, comprising:
a mask and a base that are split and capable of connecting to each other, wherein the mask is wearable on a human body and configured for acquiring wearing information of the human body, and when the mask is connected to the base, the base is able to charge the mask, the mask transmits the acquired wearing information to the base, and the base transmits the information to a cloud server.

2. The split wireless detection mask according to claim 1, wherein, the mask comprises a control unit, a detection unit, a power supply unit and a detection information transmitting unit, wherein the detection unit, the power supply unit and the detection information transmitting unit are electrically connected with the control unit respectively, the power supply unit charges the mask, and the wearing information is acquired by the detection unit.

3. The split wireless detection mask according to claim 2, wherein, the base comprises a wireless transmission unit, a power source unit and a detection information receiving unit, wherein the power source unit is connected with an external power source, the wireless transmission unit, the power source unit and the detection information receiving unit are electrically connected, and the detection information receiving unit is configured for receiving data sent by the detection information transmitting unit.

4. The split wireless detection mask according to claim 2, wherein the wearing information comprises information about whether a human body is wearing a mask, information about length of time of the mask being worn and additional information of the mask being worn.

5. The split wireless detection mask according to claim 4, wherein if the detection unit detects that a human body is wearing the mask, the control unit starts timing, and when the detection unit detects that the human body has removed the mask, the control unit stops the timing so as to detect the information about the length of time of the mask being worn by the human body.

6. The split wireless detection mask according to claim 4, wherein the additional information about the mask being worn comprises information of correction effect for characterizing the mask being used as an orthodontic apparatus, the additional information including a wearing posture, and a stress between the mask and teeth.

7. The split wireless detection mask according to claim 1, wherein a connection of the mask to the base is an electrical connection, and information transmission between the mask and the base adopts wired or wireless transmission; and the base and the cloud server achieve data transmission through a wireless transmission unit.

8. The split wireless detection mask according to claim 1, wherein a plug connector or a magnetic connector is configured as a connecting type between the mask and the base.

9. The split wireless detection mask according to claim 2, wherein the power supply unit further comprises a charging unit.

10. The split wireless detection mask according to claim 2, wherein the detection unit is a capacitive touch sensing circuit.

11. The split wireless detection mask according to claim 10, wherein the capacitive touch sensing circuit adopts a RC charge detection principle, and the capacitive touch sensing circuit comprises a plurality of resistors, a plurality of capacitors and a plurality of reserved contacts, and the reserved contacts comprise conductive metal sheets for detecting whether a human body is connected to the detection unit.

12. The split wireless detection mask according to claim 11, wherein when the human body contacts the reserved contacts to cause the capacitive touch sensing circuit to conduct, it is equivalent to connecting in a capacitor for applying a periodic charging voltage to the capacitive touch sensing circuit, and detecting a voltage across the capacitor, and when time required for the voltage across the capacitor to reach a threshold is extended, it is indicated that the human body has contacted the detection unit.

13. The split wireless detection mask according to claim 2, wherein the control unit comprises a controller, and a model of the controller is one of STM32F103C8T6 or ATmega8.

14. The split wireless detection mask according to claim 2, wherein the control unit comprises a timing unit, and the timing unit sets a fixed time, and continuously transmits a signal to the control unit periodically at the fixed time, wherein the control unit detects whether there is human contact in the detection unit, and if the human body is already in contact, the control unit starts counting by taking a transmission signal of the timing unit at a moment when the human body is in contact, and when the control unit is unable to receive a signal of the human contact in the detection unit, the transmission signal of the timing unit at this time is used as counting end.

15. The split wireless detection mask according to claim 2, wherein the control unit comprises a memory unit, the memory unit stores information detected by the detection unit, and the memory unit is a non-volatile memory unit.

16. The split wireless detection mask according to claim 3, wherein a transmission mode of the wireless transmission unit is one or more of GSM, WiFi or Bluetooth.

17. A method for acquiring wearing information of a human body utilizing the split wireless detection mask according to claim 1, wherein a detection unit in the mask is utilized to detect whether a human body is wearing the mask, a wearing length of time or additional wearing information; if the human body is already wearing the mask, the control unit starts counting and if the human body has removed the mask, the control unit stops counting, and the detection unit transmits and stores the information about the wearing length of time and the additional wearing information to the control unit; when the mask is fixedly connected with the base, data in the control unit is transmitted to the base, then to a cloud server through a wireless transmission unit in the base, achieving real-time acquisition and transmission of the wearing information of the human body.
